(19) United States
(12) Patent Application Publication
(10) Pub. No.: US 2020/0129150 A1
(43) Pub. Date: Apr. 30, 2020

(54) Ultrasonic Probe Positioning Method and Ultrasonic System

(71) Applicant: QISDA CORPORATION, Taoyuan City (TW)

(72) Inventor: Chia-Hsin Lin, Hsinchu County (TW)

(21) Appl. No.: 16/423,139

(22) Filed: May 27, 2019

(30) Foreign Application Priority Data
Oct. 25, 2018 (CN) 201811250522.1

(51) Int. Cl. A61B 8/00; G01S 7/52; A61B 8/13

(52) U.S. Cl. CPC A61B 8/4254; A61B 8/13; A61B 8/463; G01S 7/5205

(57) ABSTRACT

An ultrasonic probe positioning method includes acquiring a first positioning location inside the ultrasonic probe, acquiring a first foot of perpendicular location corresponding to the first positioning location on an image marginal line with a detecting depth of the ultrasonic probe, setting a first center location within a detecting coverage of the ultrasonic probe, acquiring a second foot of perpendicular location, a normal plane vector, and a plane equation corresponding to the normal plane vector of the ultrasonic probe after the ultrasonic probe is shifted with an offset and rotated with a rotating angle, generating a second center location satisfying the plane equation according to the plane equation and the first center location, and optionally displaying a spherical space corresponding to the second center location on an ultrasonic slice image according to a distance between the first center location and the second center location.

### BACKGROUND OF THE INVENTION

### 1. Field of the Invention

The present invention illustrates an ultrasonic probe positioning method and an ultrasonic System, and more particularly, an ultrasonic probe positioning method and an ultrasonic system capable of positioning three-dimensional coordinates of the ultrasonic probe continuously.

### 2. Description of the Prior Art

With rapid development of medical technologies, ultrasonic detection technologies are widely used for surgery and internal medicine. In general, the ultrasonic detection method uses an ultrasonic probe for emitting an ultrasonic signal below a skin. Further, the ultrasonic probe can receive a reflected ultrasonic signal for detecting a shape and a position of at least one object which is invisible to naked eyes. Therefore, the ultrasonic detection technologies can be used for various medical purposes.

Nowadays, an ultrasonic system is widely used for a medical diagnosis process, such as a biopsy process, a drainage process, and a drug injection therapy process. However, operations of the ultrasonic system have to rely on a physician's experience and their hand feelings. For example, a detection angle, a detection position, and a moving distance of the ultrasonic probe depend on the physician's experience and their hand feelings. In other words, if an inexperienced physician operates the ultrasonic probe, it may take more time to find a correct drug injecting position or a correct needle inserting position through a trial and error approach. Thus, if the conventional ultrasonic system is operated by the inexperienced physician, a prime treatment period may be missed due to time consumption. A probability of surgical failure is increased because of drug injection error or needle inserting error.

### SUMMARY OF THE INVENTION

In an embodiment of the present invention, an ultrasonic probe positioning method is disclosed. The method comprises setting a first positioning location inside the ultrasonic probe, acquiring a first foot of perpendicular location corresponding to the first positioning location on an image marginal line with a detecting depth of the ultrasonic probe, wherein a line between the first positioning location and the first foot of perpendicular location is perpendicular to the image marginal line, setting a first center location within a detecting coverage of the ultrasonic probe, acquiring a second positioning location inside the ultrasonic probe according to the first positioning location, an offset, and a rotating angle of the ultrasonic probe after the ultrasonic probe is shifted with the offset and rotated with the rotating angle, acquiring a second foot of perpendicular location inside the ultrasonic probe according to the first foot of perpendicular location, the offset, and the rotating angle of the ultrasonic probe, generating a plane derived by a normal vector according to the second positioning location and the second foot of perpendicular location, generating a second center location on the plane according to the plane and the first center location, wherein the second center location is selected from the plane by minimizing a distance between the plane and the first center location, and optionally displaying a spherical space corresponding to the second center location on an ultrasonic slice image according to a distance between the first center location and the second center location.

In another embodiment of the present invention, an ultrasonic system is disclosed. The ultrasonic system comprises an ultrasonic probe, a processor, and a display device. The ultrasonic probe is configured to detect at least one object inside a space below a surface. The processor is coupled to the ultrasonic probe and configured to process positioning data of the ultrasonic probe. The display device is coupled to the processor and configured to display an ultrasonic slice image. The processor sets a first positioning location inside the ultrasonic probe, acquires a first foot of perpendicular location corresponding to the first positioning location on an image marginal line with a detecting depth of the ultrasonic probe. A line between the first positioning location and the first foot of perpendicular location is perpendicular to the image marginal line. The processor sets a first center location within a detecting coverage of the ultrasonic probe, acquires a second positioning location inside the ultrasonic probe according to the first positioning location, an offset, and a rotating angle of the ultrasonic probe after the ultrasonic probe is shifted with the offset and rotated with the rotating angle, acquires a second foot of perpendicular location inside the ultrasonic probe according to the first foot of perpendicular location, the offset, and the rotating angle of the ultrasonic probe, generates a plane derived by a normal vector according to the second positioning location and the second foot of perpendicular location, generates a second center location on the plane according to the plane and the first center location. The second center location is selected from the plane by minimizing a distance between the plane and the first center location. The processor controls the display device for optionally displaying a spherical space corresponding to the second center location on an ultrasonic slice image according to a distance between the first center location and the second center location.

These and other objectives of the present invention will no doubt become obvious to those of ordinary skill in the art after reading the following detailed description of the preferred embodiment that is illustrated in the various figures and drawings.

### BRIEF DESCRIPTION OF THE DRAWINGS

FIG. 1 is a block diagram of an ultrasonic system according to an embodiment of the present invention.

FIG. 2 is an illustration of initializing a plurality of coordinates in a three-dimensional coordinate space of the ultrasonic system in FIG. 1.

FIG. 3 is an illustration of generating a spherical memory space centered on a first center location according to the first center location and a first radius of the ultrasonic system in FIG. 1.

FIG. 4 is an illustration of acquiring a plurality of coordinates in the three-dimensional coordinate space after the ultrasonic probe of the ultrasonic system in FIG. 1 is moved.

FIG. 5 is an illustration of generating a second center location on a plane according to the plane and a first center location after the ultrasonic probe of the ultrasonic system in FIG. 1 is moved.

FIG. 6 is an illustration of displaying a spherical space corresponding to the second center location on an ultrasonic slice image of the ultrasonic system in FIG. 1.

FIG. 7 is a flow chart of an ultrasonic probe positioning method performed by the ultrasonic system in FIG. 1.

### DETAILED DESCRIPTION

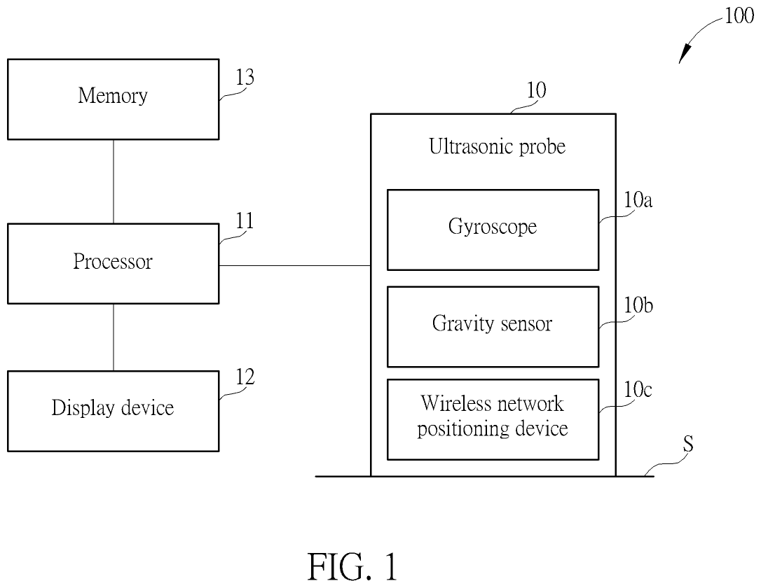

FIG. 1 is a block diagram of an ultrasonic system 100 according to an embodiment of the present invention. The ultrasonic system 100 includes an ultrasonic probe 10, a processor 11, and a display device 12. The ultrasonic probe 10 is used for detecting at least one object inside a space below a surface S. The ultrasonic probe 10 can be a curvilinear array (CLA) probe, a linear array (LA) probe, a phased array (PA) probe, or any type of probe. Further, the surface S can be a skin surface. The ultrasonic probe 10 can be used for detecting locations and shapes of bones, blood vessels, or any biological tissue or organ below the skin surface S. The ultrasonic probe 10 can include a gyroscope 10a, a gravity sensor 10b, and a wireless network positioning device 10c. After the ultrasonic probe 10 is moved, the gyroscope 10a and the gravity sensor 10b can detect a rotating angle of the ultrasonic probe 10. The wireless network positioning device 10c can detect an offset of the ultrasonic probe 10. The wireless network location device 10c can be a Bluetooth positioning device or any short or medium distance based wireless positioning device. The processor 11 is coupled to the ultrasonic probe 10 for processing positioning data of the ultrasonic probe 10. The processor 11 can be any type of processing device, such as an ultrasonic inspection machine, a computer, or an ultrasonic workstation. The ultrasonic probe 10 can be coupled to the processor 11 through a wired or wireless link. The processor 11 can be installed with an application program for synchronizing the gyroscope 10a, the gravity sensor 10b, and the wireless network positioning device 10c. The processor 11 can be coupled to an auxiliary circuit for accurately acquiring instant positioning data of the ultrasonic probe 10. The display device 12 is coupled to the processor 11 for displaying an ultrasonic slice image. The display device 12 can be any type of monochrome display device or RGB display device for displaying the ultrasonic slice image with a trapezoidal detection range of the ultrasonic probe 10. The ultrasonic system 100 can further include a memory 13. The memory 13 is coupled to the processor 11 for saving positioning data (i.e., data of positioning coordinates) and positioning function data (i.e., a plane equation and a normal vector, as illustrated later). Further, the memory 13 can be disposed on a mainboard of the processor 11. The memory 13 can also be an external memory (i.e., an external hard disk) or a memory space of a cloud server. Any reasonable hardware modification falls into the scope of the present invention. In the ultrasonic system 100, the processor 11 can set a first positioning location inside the ultrasonic probe 10. The processor 11 can further acquire a first foot of perpendicular location corresponding to the first positioning location on an image marginal line with a detecting depth of the ultrasonic probe 10. Here, a line between the first positioning location and the first foot of perpendicular location is perpendicular to the image marginal line. Then, the processor 11 can set a first center location within a detecting coverage of the ultrasonic probe 10. After the ultrasonic probe 10 is shifted by an offset and rotated with a rotating angle, the processor 11 can acquire a second positioning location inside the ultrasonic probe 10 according to the first positioning location, the offset, and the rotating angle of the ultrasonic probe 10. Then, the processor 11 can acquire a second foot of perpendicular location inside the ultrasonic probe 10 according to the first foot of perpendicular location, the offset, and the rotating angle of the ultrasonic probe 10. The processor 11 can generate a plane derived by a normal vector according to the second positioning location and the second foot of perpendicular location. Then, the processor 11 can generate a second center location on the plane according to the plane and the first center location. Particularly, the second center location is selected from the plane by minimizing a distance between the plane and the first center location. Finally, the processor 11 can control the display device 12 for optionally displaying a spherical space corresponding to the second center location on the ultrasonic slice image according to a distance between the first center location and the second center location. Details of an ultrasonic probe positioning method performed by the ultrasonic system 100 are illustrated below.

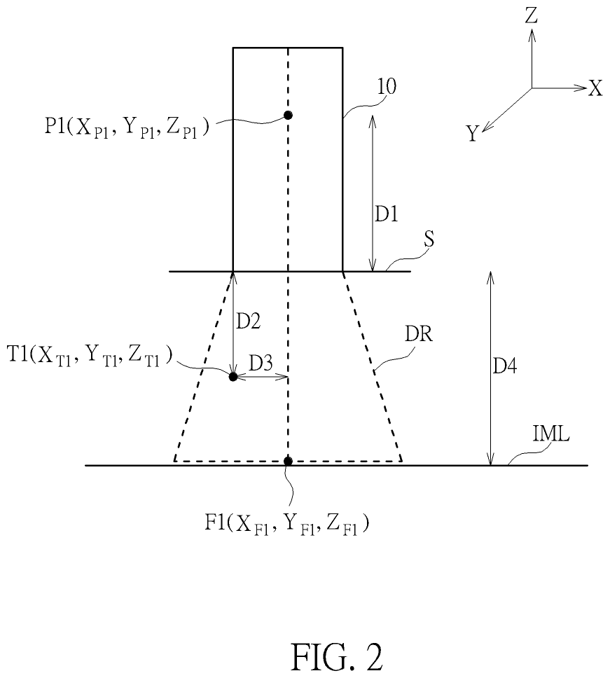

FIG. 2 is an illustration of initializing a plurality of coordinates in a three-dimensional coordinate space of the ultrasonic system 100. As shown in FIG. 2, the three-dimensional coordinate space can be a space of a Cartesian Coordinate System. A horizontal axis is denoted as an axis “X”. A vertical axis is denoted as an axis “Z”. An axis Y is perpendicular to the axis X and axis Z. When the ultrasonic system 100 is initialized, a first positioning location P1 inside the ultrasonic probe 10 can be determined. For example, coordinates of the first positioning location P1 can be denoted as P1(X_{P1}, Y_{P1}, Z_{P1}). A center line of the ultrasonic probe 10 can pass through the first positioning location P1. Further, in order to reduce computational complexity, the processor 11 can set the first positioning location P1 as an origin of the Cartesian Coordinate System after the information of the first positioning location P1 is received by the processor 11, as P1(X_{P1}, Y_{P1}, Z_{P1})=P1(0, 0, 0). Then, the processor 11 can acquire a first foot of perpendicular location F1 corresponding to the first positioning location P1 on an image marginal line IML with a detecting depth D4 of the ultrasonic probe 10. The detecting depth D4 can be defined as a detecting distance (i.e., from the surface S to inward) available for the ultrasonic probe 10. For example, the detecting depth D4 can be a user-defined value. The detecting depth D4 can also be a maximum detecting distance of the ultrasonic probe 10. Further, a line between the first positioning location P1 and the first foot of perpendicular location F1 is perpendicular to an image marginal line IML. In FIG. 2, coordinates of the first foot of perpendicular location F1 can be denoted as F1(X_{F1}, Y_{F1}, Z_{F1}). When a vertical distance between the first positioning location P1 and the surface S is equal to D1, coordinates F1(X_{F1}, Y_{F1}, Z_{F1}) of the first foot of perpendicular location F1 can be denoted as F1(0, 0, −D1−D4). In other words, the center line of the ultrasonic probe 10 can pass through the first positioning location P1. The first foot of perpendicular location F1 is at an intersection of the center line and the image marginal line IML with the detecting depth D4 of the ultrasonic probe 10. The first foot of perpendicular location F1 can also be located around the intersection of the center line and the image marginal line IML. Further, before the ultrasonic probe 10 is used for generating the ultrasonic slice image, the processor 11 can set a first center location T1 within a detecting coverage of the ultrasonic probe 10. Here, the detecting coverage of the ultrasonic probe 10 can be a trapezoidal region DR. Coordinates of the first center location T1 can be denoted as T1 (X_{T1}, Y_{T1}, Z_{T1}). Here, a vertical distance between the first center location T1 and the surface S is D2. A distance between the first center location T1 and the center line is D3. Therefore, the coordinates of the first center location T1 can be written as T1(X_{T1}, Y_{T1}, Z_{T1})=T1(−D3, 0, −D1−D2).

In FIG. 2, the coordinates of the first positioning location P1 are P1(0, 0, 0). The coordinates of the first foot of perpendicular location F1 are F1(0, 0, −D1−D4). The coordinates of the first center location T1 are T1(−D3, 0, −D1−D2). Thus, when the ultrasonic system 100 is initialized, a plane equation and its normal vector can be derived according to the first positioning location P1, the first foot of perpendicular location F1, and the first center location T1, as illustrated below.

(1) a direction vector from P1 to F1 is (0, 0, −D1−D4)
(2) a direction vector from P1 to T1 is (−D3, 0, −D1−D2)

Here, (n_{X1}, n_{Y1}, n_{Z1}) is defined as the normal vector of the plane equation corresponding to the first positioning location P1, the first foot of perpendicular location F1, and the first center location T1. The normal vector (n_{X1}, n_{Y1}, n_{Z1}) and the direction vector from P1 to F1 are orthogonal. The normal vector (n_{X1}, n_{Y1}, n_{Z1}) and the direction vector from P1 to T1 are orthogonal. As a result, −(D1+D4)×n_{Z1}=0 and −D3×n_{X1}−(D1+D2)×n_{Z1}=0 can be derived. The normal vector (n_{X1}, n_{Y1}, n_{Z1}) can be derived as (0, n_{Y1}, 0). In other words, a plane equation y=0 satisfying the first positioning location P1, the first foot of perpendicular location F1, and the first center location T1 can be derived. Equivalently, a plane of the plane equation y=0 can be regarded as an initial detection plane of the ultrasonic system 100.

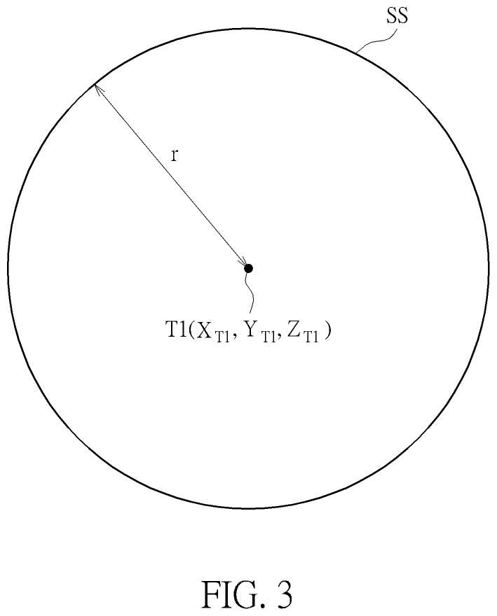

FIG. 3 is an illustration of generating a spherical memory space SS centered on a first center location T1 according to the first center location T1 and a first radius r of the ultrasonic system 100. As previously mentioned, before the ultrasonic probe 10 is used for generating the ultrasonic slice image, the processor 11 can set the first center location T1 within the detecting coverage of the ultrasonic probe 10. Then, the processor 11 can set a first radius r of a sphere centered on the first center location T1. Then, the processor 11 can generate a spherical memory space SS centered on the first center location T1. An equation of the spherical memory space SS can be written as (x−X_{T1})²+(y−Y_{T1})²+(Z−Z_{T1})²=r². Particularly, the spherical memory space SS can be regarded as a virtual three-dimensional space generated by the ultrasonic system 100. The spherical memory space SS can also be regarded as a target space or a referenced space for injecting drug by medical personnel.

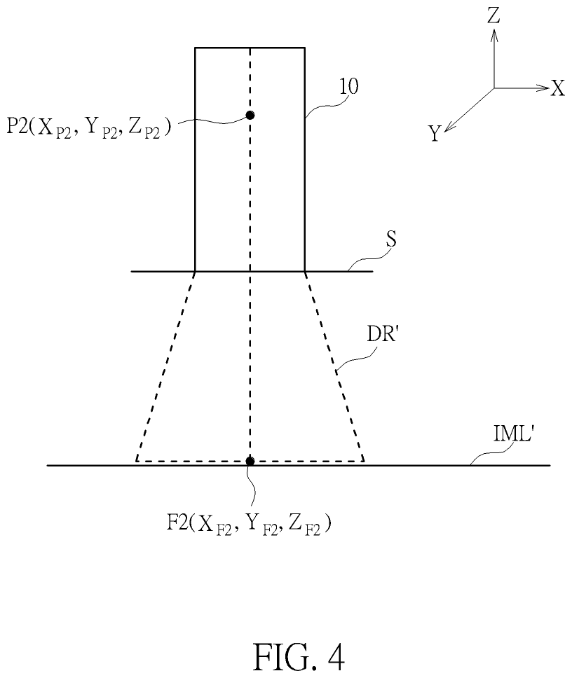

After the first positioning location P1, the first foot of perpendicular location F1, and the first center location T1 are determined, the ultrasonic probe 10 can be arbitrarily moved. FIG. 4 is an illustration of acquiring a plurality of coordinates in the three-dimensional coordinate space after the ultrasonic probe 10 of the ultrasonic system 100 is moved. The ultrasonic probe 10 includes a gyroscope 10a, a gravity sensor 10b, and a wireless network positioning device 10c. Positioning data of the gyroscope 10a, the gravity sensor 10b, and the wireless network positioning device 10c can be synchronized with the processor 11. Therefore, after the ultrasonic probe 10 is moved with a rotating angle and an offset, the gyroscope 10a, the gravity sensor 10b, and the wireless network positioning device 10c can detect the rotating angle and the offset. Further, the processor 11 can acquire a second positioning location P2 inside the ultrasonic probe 10 according to the first positioning location P1, the offset, and the rotating angle. For example, the coordinates of the first positioning location P1 are P1 (X_{P1}, Y_{P1}, Z_{P1}). After the ultrasonic probe 10 is moved, the processor 11 can acquire coordinates of the second positioning location P2, denoted as P2 (X_{P2}, Y_{P2}, Z_{P2}). Similarly, the processor 11 can acquire a second foot of perpendicular location F2 inside the ultrasonic probe 10 according to the first foot of perpendicular location F1, the offset, and the rotating angle. For example, the coordinates of the first foot of perpendicular location F1 are F1(X_{F1}, Y_{F1}, Z_{F1}). After the ultrasonic probe 10 is moved, the processor 11 can acquire coordinates of the second foot of perpendicular location F2, denoted as F2 (X_{F2}, Y_{F2}, Z_{F2}). Further, a line between the second positioning location P2 and the second foot of perpendicular location F2 is perpendicular to an image marginal line IML′ corresponding to a trapezoidal region DR′ Similarly, a center line of the ultrasonic probe 10 can pass through the second positioning location P2. The second foot of perpendicular location F2 is at an intersection of the center line and the image marginal line IML′ of the ultrasonic probe 10. Then, the processor 11 can generate a plane equation and its normal vector according to the second positioning location P2, the second foot of perpendicular location F2, the rotating angle, and the offset. For example, after the ultrasonic probe 10 is moved, the processor 11 can generate a normal vector (n_{X2}, n_{Y2}, n_{Z2}). Further, the normal vector (n_{X2}, n_{Y2}, n_{Z2}) and a direction vector from the second positioning location P2 to the second foot of perpendicular location F2 are orthogonal. In other words, the normal vector (n_{X2}, n_{Y2}, n_{Z2}) satisfies an equation of n_{X2}×(X_{F2}−X_{P2})+n_{Y2}×(Y_{F2}−Y_{P2})+n_{Z2}×(Z_{F2}−Z_{P2})=0. Here, a vector [(X_{F2}−X_{P2}), (Y_{F2}−Y_{P2}), (Z_{F2}−Z_{P2})] is denoted as a direction vector from the second positioning location P2 to the second foot of perpendicular location F2. According to the normal vector (n_{X2}, n_{Y2}, n_{Z2}), the processor can generate a plane equation, as derived below.

n_{X2}×(x−X_{P2})+n_{Y2}×(y−YP₂)+n_{Z2}×(z−Z_{P2})=0

Since the ultrasonic probe 10 is moved, the plane equation y=0 and the plane equation n_{X2}×(x−X₂)+n_{Y2}×(y−Y_{P2})+n₂×(z−Z_{P2})=0 are different. The plane equation n_{X2}×(x−X_{P2})+n_{Y2}×(y−Y_{P2})+n_{Z2}×(z−Z_{P2})=0 can be regarded as an updated plane equation.

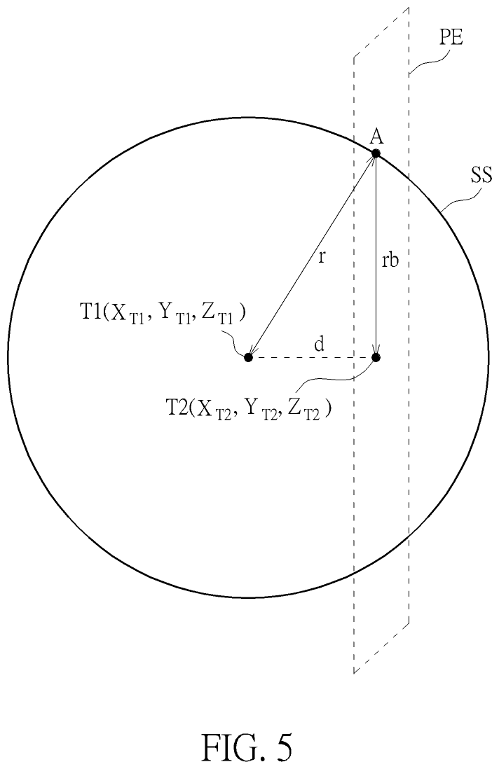

FIG. 5 is an illustration of generating a second center location T2 on a plane PE of the plane equation according to the plane PE and the first center location T1 after the ultrasonic probe 10 of the ultrasonic system 100 is moved. As previously mentioned, after the first center location T1 and the first radius r are determined, the processor 11 can generate the spherical memory space SS centered on the first center location T1. The equation of the spherical memory space SS can be written as (x−X_{T1})²+(y−Y_{T1})²+(z−Z_{T1})²=r². Further, the processor 11 can generate the plane PE according to the plane equation n_{X2}×(x−X₂)+n_{Y2}×(y−Y_{P2})+n_{Z2}×(z−Z₂)=0 after the ultrasonic probe 10 is moved. Therefore, the processor 11 can generate a second center location T2 on the plane PE of the plane equation according to the plane equation and the first center location T1. Specifically, the second center location T2 is selected from the plane PE of the plane equation by minimizing a distance between the plane PE of the plane equation and the first center location T1. In other words, the second center location T2 can be regarded as a projection point at which the first center location T1 is projected on the plane PE of the plane equation. Coordinates of the second center location T2 can be denoted as T2 (X_{T2}, Y_{T2}, Z_{T2}). Further, a distance between the second center location T2 and the first center location T1 is equal to d. When a circular section plane is generated by the plane PE of the plane equation and the spherical memory space SS, the second center location T2 can be regarded as a center of the circular section plane. Further, the processor 11 can generate a circular section radius of the circular section plane, denoted as rb. According to Pythagorean Theorem, the first center location T1, the second center location T2, and a tangent location A form a right triangle. Therefore, the first radius r can be regarded as an oblique side of the right triangle. Thus, r²=d²+rb² always holds. Further, the circular section radius rb of the circular section plane is smaller than the first radius r. Then, the processor 11 can control the display device 12 for optionally displaying a spherical space corresponding to the second center location T2 on the ultrasonic slice image according to the distance d between the first center location T1 and the second center location T2, as illustrated later.

As previously mentioned, the processor 11 can generate the second center location T2 on the plane PE of the plane equation, and detect the distance d between the first center location T1 and the second center location T2. Then, the processor 11 can generate a spherical space centered on the second center location T2 according to the second center location T2 and the circular section radius rb. The circular section radius rb is regarded as a second radius of the of the spherical space centered on the second center location T2, so that the second radius is called as the second radius rb hereafter. When the distance d between the first center location T1 and the second center location T2 is smaller than or equal to the first radius r and the spherical space with the second radius rb is within the ultrasonic slice image, the spherical space (i.e., centered on the second center location T2 and having the second radius rb) can be displayed on the ultrasonic slice image.

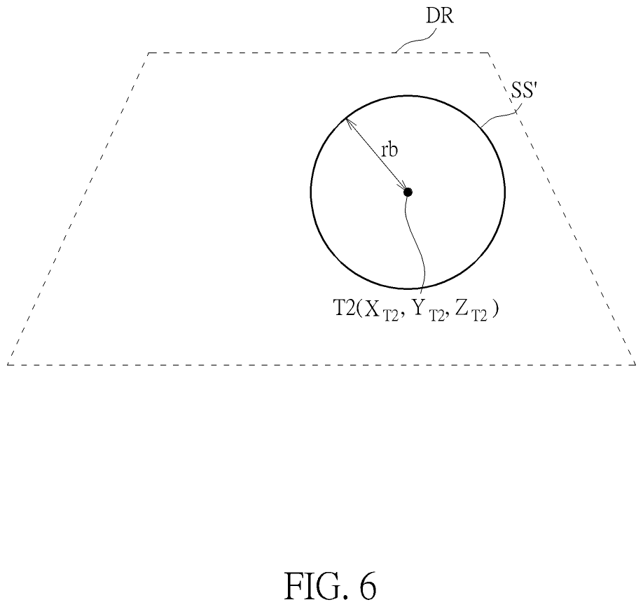

FIG. 6 is an illustration of displaying the spherical space SS' corresponding to the second center location T2 on the ultrasonic slice image of the ultrasonic system 100. The ultrasonic slice image can be the trapezoidal region DR. The spherical space SS' centered on the second center location T2 and having the second radius rb can be displayed on the ultrasonic slice image. An equation of the spherical space SS' can be written as (x−X_{T2})²+(y−Y_{T2})²+(z−Z_{T2})²=rb². Further, the spherical memory space SS of the first center location T1 and the spherical space SS' of the second center location T2 are within the ultrasonic slice image (the trapezoidal region DR). In other words, after the ultrasonic probe 10 is moved, if the detecting coverage of the ultrasonic probe 10 is not far from the spherical memory space SS (i.e., or say, a target drug injecting space or needle inserting space), the processor 11 still controls the display device 12 for displaying the spherical space SS' in order to provide positioning information to medical personnel. Therefore, the medical personnel can calibrate the rotating angle or the position of the ultrasonic probe 10 according to the positioning information. In other words, the medical personnel can quickly calibrate the ultrasonic probe 10 in order to align the target drug injecting space or needle inserting space.

Contrarily, when the distance d between the first center location T1 and the second center location T2 is greater than the first radius r or the spherical space SS' corresponding to the second center location T2 is outside the ultrasonic slice image, it implies that the ultrasonic probe 10 is far from the spherical memory space SS (i.e., or say, the target drug injecting space or needle inserting space). Therefore, the processor 11 can control the display device 12 to generate a positioning shift signal in order to stop displaying the ultrasonic slice image temporarily. By doing so, when the medical personnel notice the positioning shift signal or absence of displaying the spherical space SS′, it implies that the ultrasonic probe 10 is operated under an erroneous condition since its coordinates are severely deviated, the medical personnel can immediately correct the rotating angle and position of the ultrasonic probe 10 in order to align the target drug injecting space or needle inserting space. As previously mentioned, any hardware modification falls into the scope of the present invention. For example, the display device 12 can display a user interface (UI) for inputting coordinates (i.e., P1 (X_{P1}, Y_{P1}, Z_{P1})) of the first positioning location P1 of the ultrasonic probe 10. However, the coordinates of the first positioning location P1 can be default values or automatically generated by using the ultrasonic system 100. After the coordinates of the first positioning location P1 are received by the processor 11, the processor 11 can define the coordinates of the first positioning location P1 as an origin of the Cartesian Coordinate System. Further, the user interface can also be used for inputting coordinates (i.e., T1(X_{T1}, Y_{T1}, Z_{T1})) of the first center location T1, the detecting depth D4, the first radius r, and/or coordinates (i.e., F1(X_{F1}, Y_{F1}, Z_{F1})) of the first foot of perpendicular location F1. Particularly, the coordinates of the first center location T1, the detecting depth D4, the first radius r, and the coordinates of the first foot of perpendicular location F1 can also be default values or automatically generated by using the ultrasonic system 100. Any reasonable parameter configuration method for initializing the ultrasonic system 100 falls into the scope of the present invention. Further, the ultrasonic system 100 is capable of performing a function of positioning continuously. Therefore, all positioning information of the ultrasonic probe 10 in an initial stage and all subsequent stages can be saved in the memory 13. For example, positioning information of the first positioning location P1, the first foot of perpendicular location F1, the first center location T1, the second positioning location P2, the second foot of perpendicular location F2, the spherical memory space SS of the first center location T1, and the spherical space SS' of the second center location T2 can be saved in the memory 13. The aforementioned positioning information can be used for assisting medical personnel to control the ultrasonic probe 10. Therefore, the medical personnel can quickly calibrate the ultrasonic probe 10 in order to align the target drug injecting space or needle inserting space.

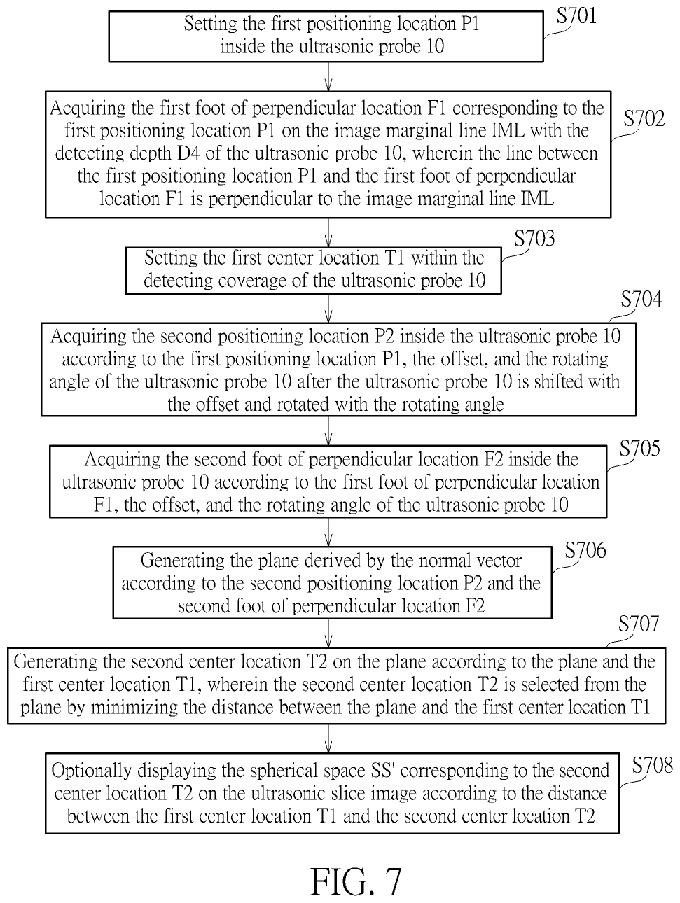

FIG. 7 is a flow chart of an ultrasonic probe positioning method performed by the ultrasonic system 100. The ultrasonic probe positioning method performed by the ultrasonic system 100 includes step S701 to step S708. Any reasonable modification falls into the scope of the present invention. Step S701 to step S708 are illustrated below.
step S701: setting the first positioning location P1 inside the ultrasonic probe 10;
step S702: acquiring the first foot of perpendicular location F1 corresponding to the first positioning location P1 on the image marginal line IML with the detecting depth D4 of the ultrasonic probe 10, wherein the line between the first positioning location P1 and the first foot of perpendicular location F1 is perpendicular to the image marginal line IML;
step S703: setting the first center location T1 within the detecting coverage of the ultrasonic probe 10;
step S704: acquiring the second positioning location P2 inside the ultrasonic probe 10 according to the first positioning location P1, the offset, and the rotating angle of the ultrasonic probe 10 after the ultrasonic probe 10 is shifted with the offset and rotated with the rotating angle;
step S705: acquiring the second foot of perpendicular location F2 inside the ultrasonic probe 10 according to the first foot of perpendicular location F1, the offset, and the rotating angle of the ultrasonic probe 10;
step S706: generating the plane derived by the normal vector according to the second positioning location P2 and the second foot of perpendicular location F2;
step S707: generating the second center location T2 on the plane according to the plane and the first center location T1, wherein the second center location T2 is selected from the plane by minimizing the distance between the plane and the first center location T1;
step S708: optionally displaying the spherical space SS' corresponding to the second center location T2 on the ultrasonic slice image according to the distance between the first center location T1 and the second center location T2.

Step S701 to step S708 are previously illustrated. Thus, details of step S701 to step S708 are omitted here. In the ultrasonic system 100, step S701 to step S703 can be regarded as initially positioning stages of the ultrasonic probe 10. Step S704 to step S708 can be regarded as continuously positioning stages of the ultrasonic probe 10. By using step S701 to step S708, the medical personnel can quickly calibrate the ultrasonic probe 10 in order to align the target drug injecting space or needle inserting space according to the spherical space SS′. When the medical personnel notice the positioning shift signal or absence of displaying the spherical space SS′, the medical personnel can immediately re-adjust (correct) the rotating angle and position of the ultrasonic probe 10.

To sum up, the present invention discloses an ultrasonic probe positioning method and an ultrasonic system. In the ultrasonic system, after the ultrasonic probe is moved, the medical personnel can confirm position accuracy of injecting drug or inserting needle according to correlation of the spherical space and the spherical memory space of the ultrasonic probe. Therefore, the ultrasonic system can be applied to a medical diagnosis process, such as a biopsy process, a drainage process, and a drug injection therapy process. Further, for the ultrasonic probe, the spherical memory space can be regarded as a target drug injection space. Thus, the medical personnel can quickly calibrate the ultrasonic probe in order to align the spherical memory space according to position of the ultrasonic probe currently detected. Further, two or more spherical memory spaces can also be introduced to the ultrasonic system for observing different lesions at different positions. In other words, the ultrasonic probe positioning method and the ultrasonic system can provide technologies for establishing virtual space (i.e., such as the spherical memory space and the spherical space) automatically. Therefore, the ultrasonic system can assist the medical personnel to deal with medical treatment.

Those skilled in the art will readily observe that numerous modifications and alterations of the device and method may be made while retaining the teachings of the invention. Accordingly, the above disclosure should be construed as limited only by the metes and bounds of the appended claims.

What is claimed is:

1. An ultrasonic probe positioning method comprising:
setting a first positioning location inside the ultrasonic probe;
acquiring a first foot of perpendicular location corresponding to the first positioning location on an image marginal line with a detecting depth of the ultrasonic probe, wherein a line between the first positioning location and the first foot of perpendicular location is perpendicular to the image marginal line;
setting a first center location within a detecting coverage of the ultrasonic probe;
acquiring a second positioning location inside the ultrasonic probe according to the first positioning location, an offset, and a rotating angle of the ultrasonic probe after the ultrasonic probe is shifted with the offset and rotated with the rotating angle;
acquiring a second foot of perpendicular location inside the ultrasonic probe according to the first foot of perpendicular location, the offset, and the rotating angle of the ultrasonic probe;
generating a plane derived by a normal vector according to the second positioning location and the second foot of perpendicular location;
generating a second center location on the plane according to the plane and the first center location, wherein the second center location is selected from the plane by minimizing a distance between the plane and the first center location; and
optionally displaying a spherical space corresponding to the second center location on an ultrasonic slice image according to a distance between the first center location and the second center location.

2. The method of claim 1, further comprising:
setting a first radius of a sphere centered on the first center location;
wherein optionally displaying the spherical space corresponding to the second center location on the ultrasonic slice image according to the distance between the first center location and the second center location, is displaying the spherical space corresponding to the second center location on the ultrasonic slice image when the distance between the first center location and the second center location is smaller than or equal to the first radius and the spherical space corresponding to the second center location is within the ultrasonic slice image.

3. The method of claim 2, further comprising:
generating a spherical memory space centered on the first center location according to the first center location and the first radius;
acquiring a circular section radius of a circular section plane formed by the plane and the spherical memory space when the second center location is a center of the circular section plane; and
generating the spherical space centered on the second center location according to the second center location and the circular section radius;
wherein a second radius of the spherical space centered on the second center location is the circular section radius, and the spherical memory space of the first center location and the spherical space of the second center location are within the ultrasonic slice image.

4. The method of claim 2, wherein the second radius is smaller than the first radius.

5. The method of claim 1, further comprising:
setting a first radius of a sphere centered on the first center location;
wherein optionally displaying the spherical space corresponding to the second center location on the ultrasonic slice image according to the distance between the first center location and the second center location, is generating a positioning shift signal to stop displaying the ultrasonic slice image temporarily when the distance between the first center location and the second center location is greater than the first radius or the spherical space corresponding to the second center location is outside the ultrasonic slice image.

6. The method of claim 1, further comprising:
detecting the rotating angle of the ultrasonic probe by using a gyroscope and a gravity sensor disposed inside the ultrasonic probe after the ultrasonic probe is moved; and
detecting the offset of the ultrasonic probe by using a wireless network positioning device after the ultrasonic probe is moved.

7. The method of claim 1, wherein the first positioning location, the first foot of perpendicular location, the first center location, the second positioning location, and the second foot of perpendicular location are corresponding to a plurality of coordinates of a Cartesian Coordinate System, and the first positioning location is an origin of the Cartesian Coordinate System.

8. The method of claim 1, wherein an inner product of the normal vector and a vector between the second positioning location and the second foot of perpendicular location is zero.

9. The method of claim 1, wherein a center line of the ultrasonic probe passes through the first positioning location, and the first foot of perpendicular location is at an intersection of the center line and the image marginal line with the detecting depth of the ultrasonic probe.

10. An ultrasonic system comprising:
an ultrasonic probe configured to detect at least one object inside a space below a surface;
a processor coupled to the ultrasonic probe and configured to process positioning data of the ultrasonic probe; and
a display device coupled to the processor and configured to display an ultrasonic slice image;
wherein the processor sets a first positioning location inside the ultrasonic probe, acquires a first foot of perpendicular location corresponding to the first positioning location on an image marginal line with a detecting depth of the ultrasonic probe, a line between the first positioning location and the first foot of perpendicular location is perpendicular to the image marginal line, the processor sets a first center location within a detecting coverage of the ultrasonic probe, acquires a second positioning location inside the ultrasonic probe according to the first positioning location, an offset, and a rotating angle of the ultrasonic probe after the ultrasonic probe is shifted with the offset and rotated with the rotating angle, acquires a second foot of perpendicular location inside the ultrasonic probe according to the first foot of perpendicular location, the offset, and the rotating angle of the ultrasonic probe, generates a plane derived by a normal vector according to the second positioning location and the second foot of perpendicular location, generates a second center location on the plane according to the plane and the first center location, the second center location is selected from the plane by minimizing a distance between the plane and the first center location, and the processor controls the display device for optionally displaying a spherical space corresponding to the second center location on an ultrasonic slice image according to a distance between the first center location and the second center location.

11. The system of claim 10, wherein the processor sets a first radius of a sphere centered on the first center location, and the processor controls the display device for displaying the spherical space corresponding to the second center location on the ultrasonic slice image when the distance between the first center location and the second center location is smaller than or equal to the first radius and the spherical space corresponding to the second center location is within the ultrasonic slice image.

12. The system of claim 11, wherein the processor generates a spherical memory space centered on the first center location according to the first center location and the first radius, acquires a circular section radius of a circular section plane formed by the plane and the spherical memory space when the second center location is a center of the circular section plane, generates the spherical space centered on the second center location according to the second center location and the circular section radius, a second radius of the spherical space centered on the second center location is the circular section radius, and the spherical memory space of the first center location and the spherical space of the second center location are within the ultrasonic slice image.

13. The system of claim 12, wherein the second radius is smaller than the first radius.

14. The system of claim 10, wherein the processor sets a first radius of a sphere centered on the first center location, the processor controls the display device to generate a positioning shift signal in order to stop displaying the ultrasonic slice image temporarily when the distance between the first center location and the second center location is greater than the first radius or the spherical space corresponding to the second center location is outside the ultrasonic slice image.

15. The system of claim 10, wherein the ultrasonic probe comprises a gyroscope, a gravity sensor, and a wireless network positioning device, after the ultrasonic probe is moved, the gyroscope and the gravity sensor detect the rotating angle of the ultrasonic probe, and the wireless network positioning device detects the offset of the ultrasonic probe.

16. The system of claim 10, wherein the first positioning location, the first foot of perpendicular location, the first center location, the second positioning location, and the second foot of perpendicular location are corresponding to a plurality of coordinates of a Cartesian Coordinate System, and the first positioning location is an origin of the Cartesian Coordinate System.

17. The system of claim 10, wherein an inner product of the normal vector and a vector between the second positioning location and the second foot of perpendicular location is zero.

18. The system of claim 10, further comprising:
a memory coupled to the processor and configured to save data of the first positioning location, the first positioning location, the first center location, the second positioning location, the second foot of perpendicular location, the second center location, a spherical memory space corresponding to the first center location, and the spherical space corresponding to the second center location.

19. The system of claim 10, wherein the display device displays a user interface (UI) for inputting coordinates information of the first positioning location of the ultrasonic probe, and the processor sets the first positioning location as an origin of a Cartesian Coordinate System after the information of the first positioning location is received by the processor.

20. The system of claim 10, wherein a center line of the ultrasonic probe passes through the first positioning location, and the first foot of perpendicular location is at an intersection of the center line and the image marginal line with the detecting depth of the ultrasonic probe.
